(19) United States
(12) Patent Application Publication
(10) Pub. No.: US 2009/0318818 A1
(43) Pub. Date: Dec. 24, 2009

(54) BLOOD PRESSURE MONITORING SYSTEM

(75) Inventors: Tyson B. Whitaker, Arden, NC (US); Matthew J. Kinsley, Liverpool, NY (US); John A. Lane, Weedsport, NY (US); Scott A. Martin, Warners, NY (US); Thomas J. Grant, Skaneateles, NY (US); Stephen F. Meyer, Skaneateles, NY (US)

Correspondence Address: HISCOCK & BARCLAY, LLP, ONE PARK PLACE, 300 S. STATE ST., SYRACUSE, NY, 13202, US

(73) Assignee: Welch Allyn, Inc., Skaneateles Falls, NY (US)

(21) Appl. No.: 12/142,866

(22) Filed: Jun. 20, 2008

(51) Int. Cl. A61B 5/02; A61B 5/00

(52) U.S. Cl. 600/495; 600/490; 600/485; 600/561

(57) ABSTRACT

A blood pressure monitoring system that includes a compact housing that contains a pneumatic circuit that is removably attached to an inflatable cuff by a hoseless connector so that the cuff can be inflated and deflated to provide blood pressure readings that are detected by a pressure sensor. A controller is also contained in the housing which controls the circuit activities and records blood pressure related data. The controller is linked via a bidirectional communication system with a processor that is contained in a host station which programs the controller and collects and records blood pressure related data. The communication system also allows the monitoring system to communicate over a wider network with other remote stations.

### FIELD OF THE INVENTION

This invention relates to a blood pressure device and, in particular, to a blood pressure system that provides a flexible and low cost platform for automatically performing a non-invasive procedure without the need of external interconnections such as pneumatic tubes and electrical wiring.

### BACKGROUND OF THE INVENTION

Non-invasive blood pressure devices generally utilize an inflatable cuff to measure a patients blood pressure. The cuff is affixed to a limb over an artery and is then inflated to constrict the flow of blood through the artery. Blood passing through the constrained vessel produces changes in the cuff pressure which can be measured by a pressure sensor to give an indication of the condition of the patients heart. Two pressures of specific interest are the systolic pressure and the diastolic pressure. Systolic pressure is the maximum arterial pressure produced during the contraction of the left ventricle of the heart. The diastolic pressure is the minimum pressure that is produced during the relaxation period during which the ventricles are filling with blood. Using the well known oscillometric method, the blood pressure cuff is initially inflated to a pressure that is higher than the expected systolic reading and the cuff is then deflated to a pressure that is lower than that of the expected diastolic reading. As the cuff pressure falls to a pressure near the systolic pressure, the pressure sensor that monitors the arterial pressure detects oscillations that are superimposed on the falling cuff pressure. Correspondingly, when the cuff pressure falls below the diastolic pressure the oscillations are no longer detected. These two events can be recorded by the sensor to provide valuable information relating to the patients heart as well as the patients heart rate.

Blood pressure devices are presently available that will automatically provide accurate blood pressure readings and, to a large extent, eliminate or minimize human error. These prior art devices, however, tend to be rather large and have external tubing and electrical wires interconnect the various components of the system. The monitoring system generally is located at a fixed location and the patient must come to the system at that specific location. Some automatic systems, particularly those found in health care facilities where many of the patients are not ambulatory, are mounted upon mobile carriers which permit the system to be wheeled between patient locations. However, because of the number of tubes and wire involved, the transporting of these systems can be difficult and the tubes and wires tend to become entangled with other fixtures and furniture. In addition, these systems usually contain a single cuff that is exchanged between patients and cross contamination is thus an ever present concern.

### SUMMARY OF THE INVENTION

It is a primary object of the present invention to improve non-invasive blood pressure devices and, in particular, monitors that are capable of automatically taking a patients blood pressure.

A further object of the present invention is to eliminate the need of sharing a common blood pressure cuff between a number of patients.

Another object of the present invention is to eliminate the need for loose pneumatic and electrical lines running between the components of an automatic blood pressure monitor.

These and other objects of the current invention are attained by a blood pressure device that includes a compact housing that contains a pneumatic circuit that is attached to an inflatable cuff by a hoseless releasable connection so that the cuff can be automatically inflated and deflated to provide blood pressure readings that are detected by a pressure sensor located in the housing. A controller is also contained in the housing which controls the activity of the pneumatic circuit and which is capable of recording and storing blood pressure reading. The controller is linked via a wireless bi-directional communication system with a processor contained within a host station which programs the controller and processes blood pressure data generated by the pressure sensor. The communication system further enables the host station to communicate with other remote stations.

### BRIEF DESCRIPTION OF THE DRAWINGS

For a better understanding of these and other objects of the invention reference will be made to the following detailed description of the invention which is to be read in conjunction with the accompanying drawings, wherein:

FIG. 1 is a perspective view illustrating a non-invasive blood pressure cuff and a compact housing contains a number of components of the present blood pressure monitoring system wherein the monitor housing is shown separated from the cuff prior to the two units being connected.

FIG. 2 is a further perspective view illustrating the bottom face of the housing and showing in further detail one member of a hoseless connection for co-joining the housing and the cuff;

FIG. 3 is an enlarged perspective view of the compact housing shown in FIG. 1 with the top cover being removed as well as a printed circuited board which is shown inverted and to the left of the open bay of the housing; and

FIG. 4 is a flow diagram schematically illustrating the relation component parts of the present blood pressure monitoring system.

### DETAILED DESCRIPTION

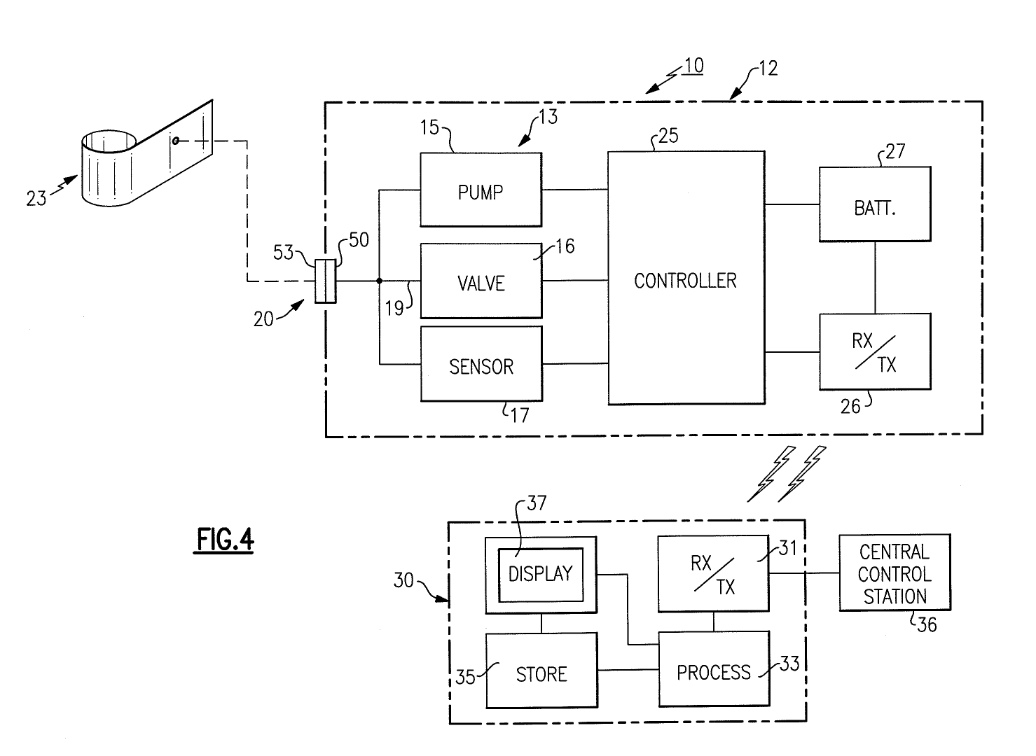

A blood pressure system, generally referenced 10, that embodies the present invention is shown schematically in FIG. 4. The system 10 includes a small compact housing 12 that contains a three element pneumatic circuit 13. The pneumatic circuit includes a pump 15, an exhaust valve 16 and a pressure sensor 17. The elements of the pneumatic circuit are all connected to a common pneumatic line 19 which, in turn, is coupled to one part 50 of a two piece connector that is generally referenced 20. As will be explained in further detail below, the other part of the connector 53 is attached to a non-invasive blood pressure cuff 23.

Also contained in the housing 12 is a controller 25 which is a microprocessor that can be programmed to control the pump and valve to inflate and deflate the cuff and to receive pressure related data from the pressure sensor. The controller is coupled to a wireless bi-directional communication unit 26 which links the controller to a remote host station that is generally referenced 30. A rechargeable battery 27 is located within the housing 12 and is arranged to power the system components situated within the housing.

The host station contains a second bi-directional communication unit 31 that is capable of linking the controller to a processor 33 that is contained within the host station. The processor is arranged to program the controller so as to carry out a desired blood pressure monitoring cycle via the pneumatic circuit 13. The processor also includes a storage unit 35 for storing blood pressure related data as well as patient related information and the time and date of various occurrences.

A display unit 37 is also contained within the host station for displaying real time blood pressure related data from the processor or historical patient related data that can be recalled from the storage unit 35. Stored or real time data can also be broadcast by bi-directional communication unit 31 to other remote stations such as a central control station 36 for further evaluation or processing of related data.

The compact housing 12, because of the limited number of compartments that are contained therein, can be extremely small in size and light of weight thus allowing it to be directly connected to and carried upon a blood pressure cuff. In addition, the connector 20, through which the pneumatic circuit communicates with a cuff, is a releasable device and accordingly, a single housing can be used in association with a number of cuffs within a heath care facility, physician's office, or the like. In this type of location, each patient can be issued his or her cuff upon entering the facility and the attendant health care worker can simply connect the monitor housing to the individual cuffs when taking blood pressure readings. As can be seen, all readings will then be sent to a central host station for processing, evaluation and storage. This procedure will reduce the risk of cross contamination that is normally present in facilities where a single cuff is used continuously to take the pressure readings of a large number of patients.

The present monitoring system can also be used in physicians offices where the patients typically must wait to be seen in a waiting area. Here, the waiting patients can be issued cuffs and a single monitor housing exchanged between patients to provide blood pressure data to the host station that will be available to the examining physician immediately upon seeing a patient. This procedure can save many hours of each day both of the physicians time and the patients time in a typical office setting.

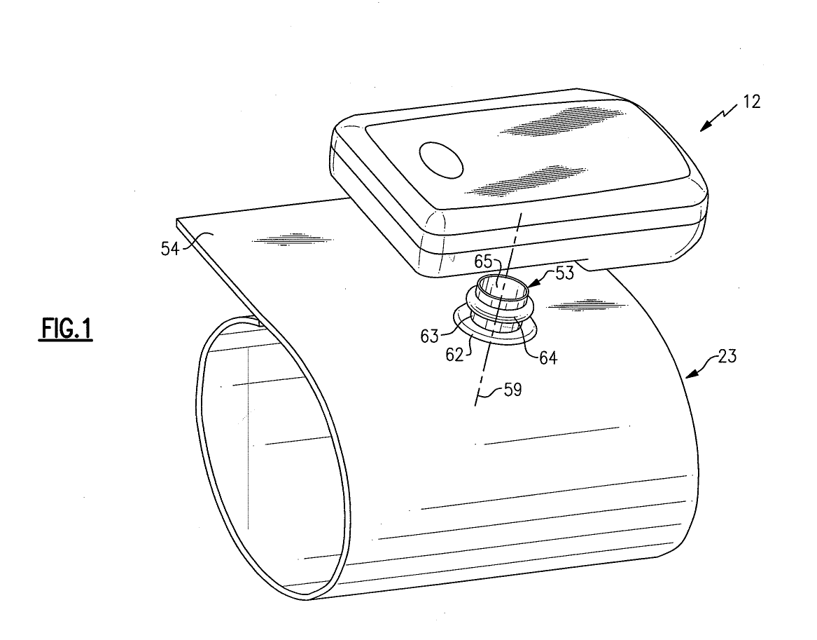

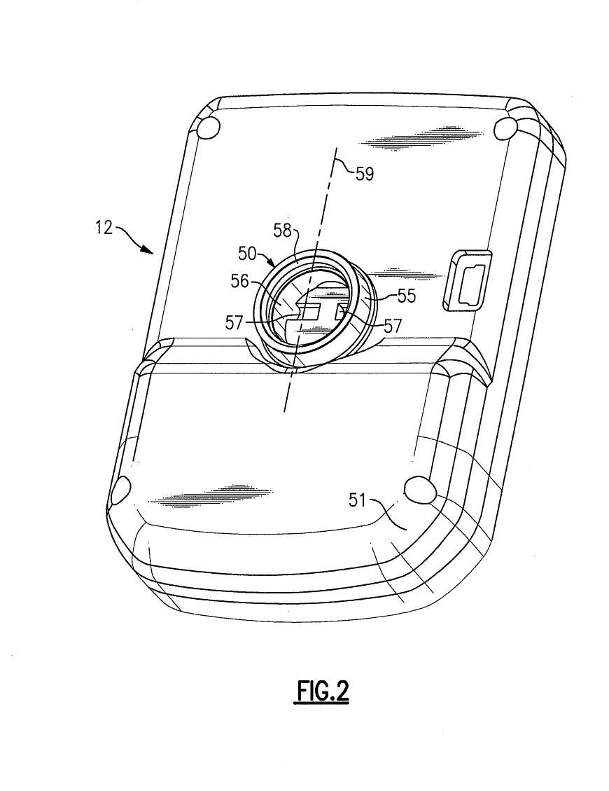

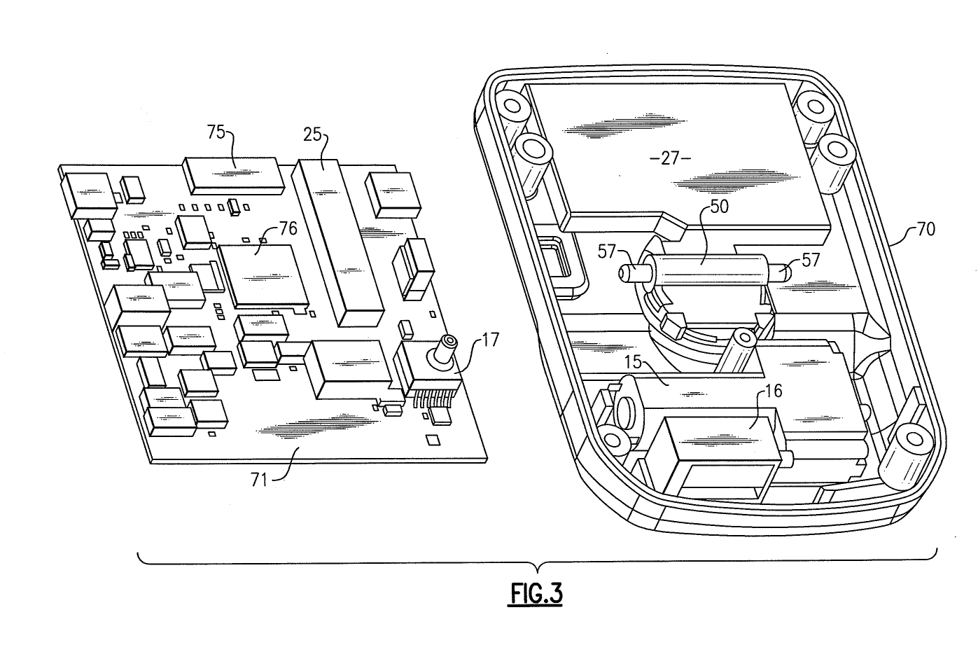

Turning now to FIGS. 1-3, there is illustrated in further detail, a monitor housing 10. As noted above, the housing is compact and is about 4.0″ high, 2.5″ wide and has a depth of about 1.0″. This coupled with the light weight of the limited number of components that are contained within the housing, permits the housing to be easily and safely attached to the cuff by means of the two piece connector 20. As illustrated in FIGS. 2 and 3, the connector 20 includes a female member 50 that is secured to the back panel 51 of the monitor housing 12. The female member is adapted to mate with a male member 53 that is secured to the sleeve 54 of a non-invasive blood pressure cuff, as for example cuff 23 (FIG. 4). As will be explained in further detail below, the male member is capable of being snap fitted into the female member to establish a releasable leak tight joint between the co-joined members.

As illustrated in FIG. 2 the female member 50 includes a cylindrical or tubular sleeve 55 that opens through the back panel of the housing into a flow channel 56 that contains a pair of entry ports 57-57 that are in fluid flow communication with the pneumatic circuit 13 (FIG. 4) via the previously noted common flow line 19. A circular groove 58 is formed in the interior wall of the tubular sleeve so that the sleeve and the groove share a common axial centerline 59.

In FIG. 1, the housing 12 is shown positioned directly over the sleeve 54 of cuff 23 just prior to closure o the two piece connector 20. At this time, the male member 53 of the connector is axially aligned with the female member of the connector along a common center line 59. The male member 53 contains a cylindrical body 63 that extends upwardly from a grommet that secures the body to the cuff sleeve. A circular bead 64 surrounds the body of the male member which snap fits into the circular groove in the female at closure. One or both of the connector members may be fabricated of a resilient material such that the joint formed between the two members at closure is leak tight but flexible enough to allow the housing to be rotated about the common axis 59 without breaking the joint. At closure, the cylindrical opening 65 in the male member is in direct fluid flow communication with the pneumatic circuit via flow ports 57-57 and flow line 19.

FIG. 3 shows the lower portion or pan 70 of the housing with the cover and the printed circuit board 71 removed from the pan so that most of the components that are contained in the housing can be more clearly viewed. The backside of the female connector 50 can be seen in the mid-section of the pan. The two flow ports 57 and 57 are shown extending to either side of the connector body. The tubing for connecting the ports to the pneumatic circuit have been removed for the sake of clarity. Immediately beneath the connector is the pump 15 and the exhaust valve 16. Above the connector 50 is the rechargeable battery 27. The communication system transmitter 75 and receiver 76 are mounted upon the backside of the circuit board 71 along with the controller 25 and the pressure sensor 17.

As can be seen, the pneumatic components and related drive and electrical components of the present invention are all tightly packaged inside the housing to provide a compact unit that can be suspended from a blood pressure cuff. As should be evident from the disclosure above, the present system has eliminated the need for troublesome electrical and pneumatic lines running between various components of the system and reduces the danger of cross contamination between patients that are forced to share a common blood pressure cuff.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

1. A non-invasive blood pressure system that includes:
a compact housing that contains a pneumatic circuit for inflating and deflating a blood pressure cuff and a pressure sensor for reading said cuff pressure and providing an output signal indicative of said cuff pressure;
a hoseless connector for removably attaching said pneumatic circuit to said cuff whereby cuff can be inflated and deflated to provide cuff pressure readings;
a programmable controller contained in said housing for controlling the components of said pneumatic circuit to provide blood pressure readings;
a remote host station that contains a processor for programming said controller and processing said cuff pressure readings; and
a bi-directional wireless communication unit for linking said processor with said controller and said pressure sensor.

2. The system of claim 1, wherein said hoseless connector includes a male member that is releasably coupled to a female member wherein one of said members is secured to said housing and the other of said members is secured to said cuff.

3. The system of claim 3, wherein said female member is secured to said housing and said male member is secured to said cuff.

4. The system of claim 3, wherein said male member is rotatably contained within said female member at closure of said connector.

5. The system of claim 3, wherein said female member contains a circular opening and said male member contains a cylindrical body.

6. The system of claim 5, wherein said circular opening of said female member contains a circular groove and said body of said male member contains a circular bead that snaps into said groove at closure.

7. The system of claim 1, wherein said pneumatic circuit contains a pump and a discharge valve that are coupled to the hoseless connector along with said pressure sensor by a common flow line.

8. The system of claim 7, wherein said processor contains a memory for storing said pressure readings.

9. The system of claim 1, wherein said housing further contains a battery for powering said pneumatic circuit components and said pressure sensor.

10. The system of claim 1, wherein said wireless bi-directional unit contains further network means for broadcasting processed pressure related data.

11. The system of claim 1, wherein said host station contains a readout for visually displaying processed pressure related data.

12. A method of monitoring a patients blood pressure that includes the steps of:
applying an inflatable blood pressure cuff to a patients limb;
establishing a hoseless connection between a said cuff and a pneumatic circuit for inflating and deflating said cuff;
measuring said cuffs pressure during an inflation and deflation cycle;
wirelessly communicating said pressure measurements to a remote host station; and
processing said measurements at said remote station.

13. The method of claim 12, that includes the further step of storing said processed measurements at said remote station.

14. The method of claim 13 that includes the further step of broadcasting said pressure measurements to one or more further remote stations.

15. The method of claim 12, that includes the further step of visually displaying said measurement at said host station.
